(19) United States
(12) Patent Application Publication
(10) Pub. No.: US 2022/0110722 A1
(43) Pub. Date: Apr. 14, 2022

(54) LIP ENHANCEMENT DEVICE

(71) Applicant: Azita NAGHAVI, Brookville, NY (US)

(72) Inventor: Azita NAGHAVI, Brookville, NY (US)

(21) Appl. No.: 17/497,004

(22) Filed: Oct. 8, 2021

Related U.S. Application Data
(60) Provisional application No. 63/089,642, filed on Oct. 9, 2020.

(51) Int. Cl. A61C 9/00; A61C 13/34; A61K 6/90

(52) U.S. Cl. CPC A61C 9/0006; A61K 6/90; A61C 13/34; A61C 9/0053

(57) ABSTRACT

A device for enhancing the appearance of a person's lips is formed from a curved member configured to fit between the person's upper or lower lip and an adjacent gum at the front of the mouth. The curved member has a gingival portion configured to be placed on the person's gums, a top surface and an extension connected to the top surface and extending from a longitudinal edge of the top surface to overlap and extend beyond the gingival portion. The extension is configured to rest against and evert the person's upper or lower lip when the device is placed between the lip and the person's gum.

### CROSS-REFERENCE TO RELATED APPLICATIONS

This application claims priority under 35 USC 119(e) of U.S. Provisional Application Ser. No. 63/089,642, filed on Oct. 9, 2020, the disclosure of which is herein incorporated by reference.

### BACKGROUND OF THE INVENTION

### 1. Field of the Invention

This invention relates to a device for insertion into a person's mouth to give the appearance of fuller lips. In particular, the invention relates to a device or a pair of devices that are held between the person's gums and lip to force the lip to protrude and give the appearance of fullness.

### 2. The Prior Art

The appearance of full lips is often considered a desirable trait, and there is a large business in providing this appearance via cosmetic procedures, such via collagen injections. However, such injections are expensive, invasive, and not without risk. Several attempts have been made to create an appliance that is insertable in the person's mouth to create the appearance of fuller lips. However, these devices are often uncomfortable and create an unnatural appearance. It would be desirable to provide a temporary, non-invasive, non-surgical way to give the appearance of fuller lips that is both comfortable to wear and gives the appearance of naturally full lips.

### SUMMARY OF THE INVENTION

It is therefore an object of the invention to provide a device for giving the appearance of fuller lips that does not involve injections or surgery. It is another object of the invention to provide such a device that is comfortable to wear, easily insertable and removable, and simple to manufacture.

These and other objects are accomplished by a lip enhancing device comprising a curved member configured to fit between the upper or lower lip and the adjacent gingiva at the front of the mouth. The curved member has a gingival portion configured to be placed on a user's gums, and an extension connected to the gingival portion and extending from a longitudinal edge of the gingival portion to overlap and extend beyond the gingival portion. In use, the extension rests against the inner portion of the lip to evert the lip and give the appearance of additional fullness. The device is held in place by friction and/or suction, or adhesive could be used. The extension is spaced from the surface of the gingival portion and can be configured to be flexible, so that it can flex with movement of the user's lips. The extension smoothly extends the upper lip, smoothing the perioral lines and the nasolabial fold areas. The device could also be used on the lower lip as well. The gingival portion can be configured to be flexible or rigid, and can be reinforced with rigid materials to preserve its shape.

The device can be made of thermoplastic nylon resin such as what is sold under the trademark VALPLAST®, or any other suitable material. The device could be made with an overall smooth surface or with small cup-like indentations or apertures for added retention. This appliance can be held in place through the suction at the muccobuccal fold or with the use of an adhesive or dental impression material such as polyvinyl siloxane, which cures quickly after mixing. By using a sufficient amount of impression material, the device can be configured to fit any mouth shape. The device can have different angulations and contours that will determine the desired displacement of the lip.

These methods of achieving that youthful look are an alternative to fillers and BOTOX® cosmetic, which have many negative side effects. There will be many samples available for the consumer to choose from in a pre-assembled kit allowing the desired fullness of the lips, nasolabial area support and cheek area.

The device could be formed so as to fit most mouth shapes, especially when used with dental impression material as the adhesive, or can be custom-molded for each user. In the custom device, the device can have a portion on each end that adheres to the buccolingual surfaces of the maxillary first and second molars, which then extends into the muccobuccal area with an angulation raising the upper lip extending out from the muccobuccal area. The emphasized areas will reflect the areas of the patient's needs to smooth the perioral and or nasolabial folds. This can be made of a variety of materials such as nylon resin, thermoformable plastic such as ESSIX C+® or any other suitable dental material.

This custom-made appliance can be fabricated by taking an impression of the maxillary and mandibular dentition and the muccobuccal areas. Alternatively, the appliance can be rendered using a virtual scan of the dentition and the muccobuccal areas. This appliance can be fabricated utilizing any suitable dental material that adheres to the muccobuccal areas and dentition or embrasure spaces. Using this appliance, a desired lip and peri-oral support can be achieved by displacing the peri-oral tissue. Depending on the circumstances, the appliance could provide minimal, moderate or maximum support as desired by the user.

An impression using alginate, a more stable impression material such as PVS (polyvinyl siloxane) or other dimensionally stable impression material can be used to model the dentition and muccobuccal area. This model is poured with dental casting stone to replicate the desired areas. Alternatively, this can be achieved with a scan of the dentition and the muccobuccal area. The desired measure of varying thickness is incorporated supra-gingivally providing support and build up to the upper and lower lip. The intramural appliance will affect the soft tissue extraorally. The thicker the appliance, the fuller the appearance of the lip.

The natural anatomy of desired areas can be built up and extended to the desired position utilizing built up plaster or potty material. After completion of this step, the Drufomat machine or another scan pressure machine can be utilized to manufacture the appliance. The material is heated and goes over the cast to create the appliance and the edges are trimmed as needed. The appliance can be fabricated to anchor around the second premolar and the last available molar. It can cover the facial aspect of the dentition as to leave the occlusal surface uncovered to prevent offset of the occlusion. Anchoring can vary with the dentition present if additional support is needed. The appliance will connect to the dentition for anchorage and emanate upwards to extend into areas where support is desired.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a first embodiment of the lip enhancing device according to the invention;

FIG. 2 show a top view of the lip enhancement device;

FIG. 3 shows a bottom view of the lip enhancement device;

FIG. 4 shows a cross-sectional view along lines IV-IV of FIG. 1;

FIG. 5 shows a front view of the lip enhancement device;

FIG. 6 shows a rear view of the lip enhancement device;

FIG. 7 shows the lip enhancement device in use on a person;

FIG. 8 shows another embodiment of the lip enhancement device according to the invention; and

FIG. 9 shows a top view of the lip enhancement device of FIG. 8.

### DETAILED DESCRIPTION OF THE PREFERRED EMBODIMENTS

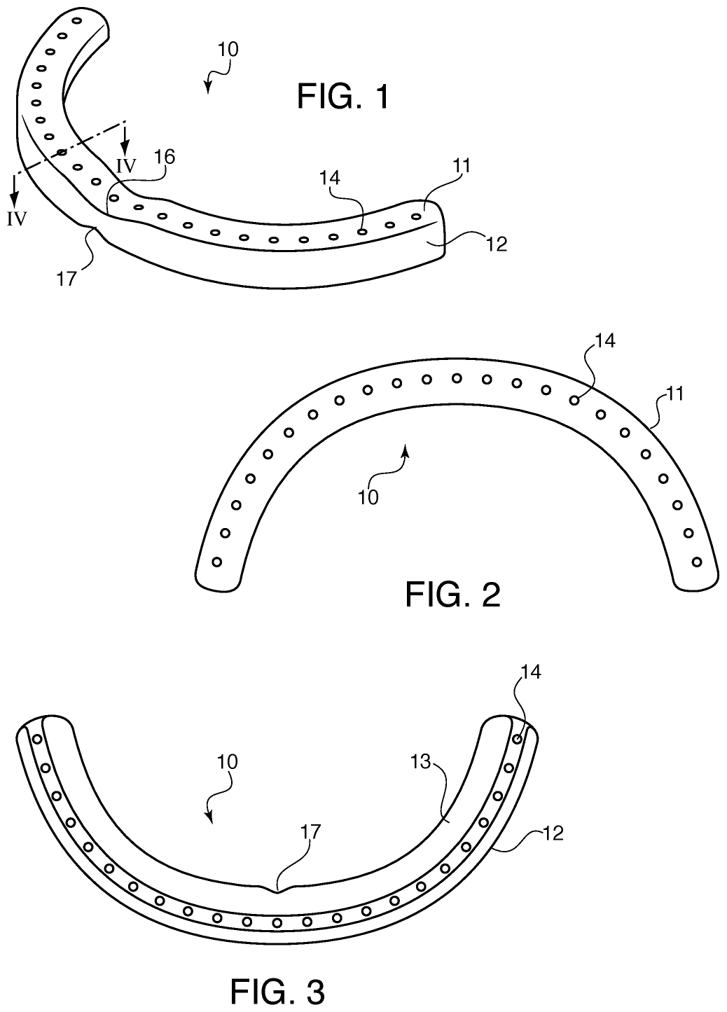

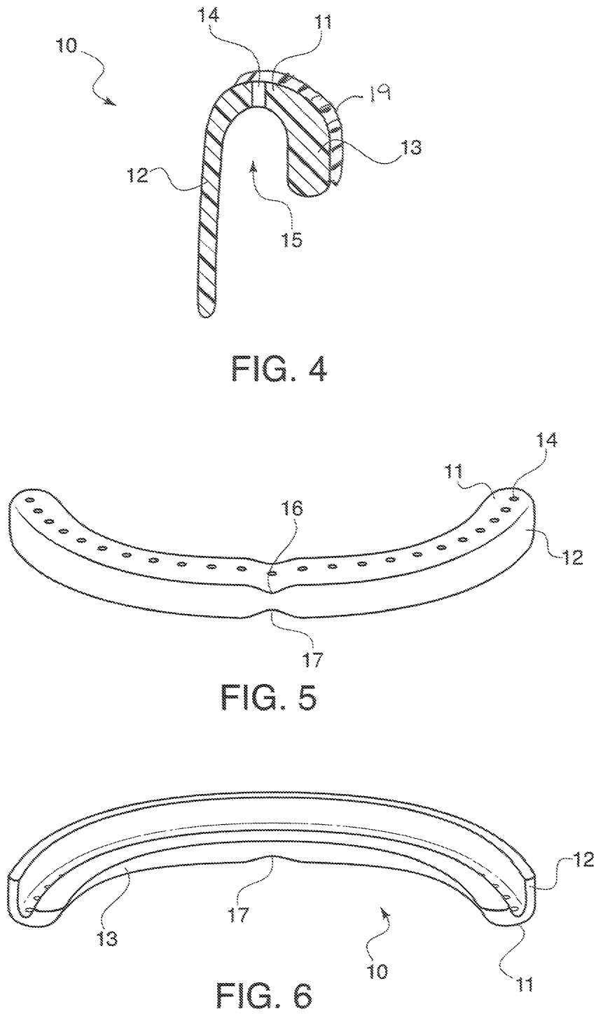

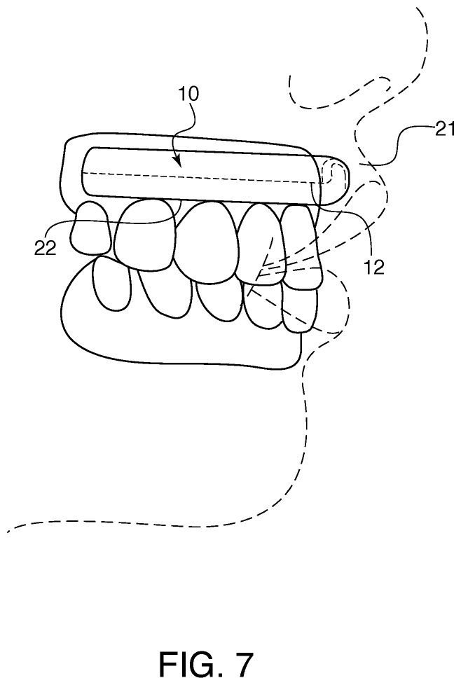

Referring now in detail to the drawings, FIGS. 1-6 show a first embodiment of the lip enhancement device 10 according to the invention. Lip enhancement device 10 is a U-shaped member having a top surface 11, a rear rounded body 13 and a front extension 12 that extends down and overlaps rear rounded body 13 with a cavity 15 in between. Front extension 12 is made so as to be thinner and taller than rear rounded body 13, and has some flexibility. The center front portion of front extension 2 has curved indentations 16, 17 on the top and bottom, so as to follow the contours of the wearer's lips and gums. The top surface 11 of lip enhancement device 10 is provided with a series of perforations 14, extending along the length of top surface 11. These perforations 14 increase the suction of lip enhancement device against the user's lips when lip enhancement device 10 is worn by the user, such as shown in FIG. 7. In FIG. 7, the lip enhancement device 10 is placed between the gums 22 and the upper lip 21, which causes the upper lip 21 to protrude outward beyond its normal position. The lip enhancement device 10 is held in place via natural suction, but an adhesive could also be applied to increase the adhesion to the user's mouth, so that the device does not move during speech or eating. As shown in FIG. 4, using dental impression material 19 applied to the top surface 11 and/or rounded body 13 can assist in creating a custom fit for the device as well as assisting in adhering the device to the gum. Once the impression material is pressed along the gum and cured, the device is essentially custom fit to the person.

The lip enhancement device 10 can be made of any suitable moldable orthodontic or dental material, such as thermoplastic nylon resin. Ideally, the device would be made of a material that is rigid enough to hold its shape while being worn, but provides flexibility to the front extension during use, so that when the user is speaking, the front extension can flex with the movement of the user's lips.

The device shown in FIGS. 1-6 can be pre-made for use with any customer, and can come in various pre-set sizes to fit various sizes of facial anatomy, and various degrees of lip enhancement, especially when used with a dental impression material to enhance the fit. In an alternative embodiment, the device can be custom made for each person, by taking a scan or making a model of the person's teeth, gingiva and lips and then forming the device to fit the person's mouth exactly, while adding to the angle of the front extension to extend the lip outwardly to the desired extent. The device could be used on only the upper lip, or two devices can be used, one on the upper lip and one on the lower lip.

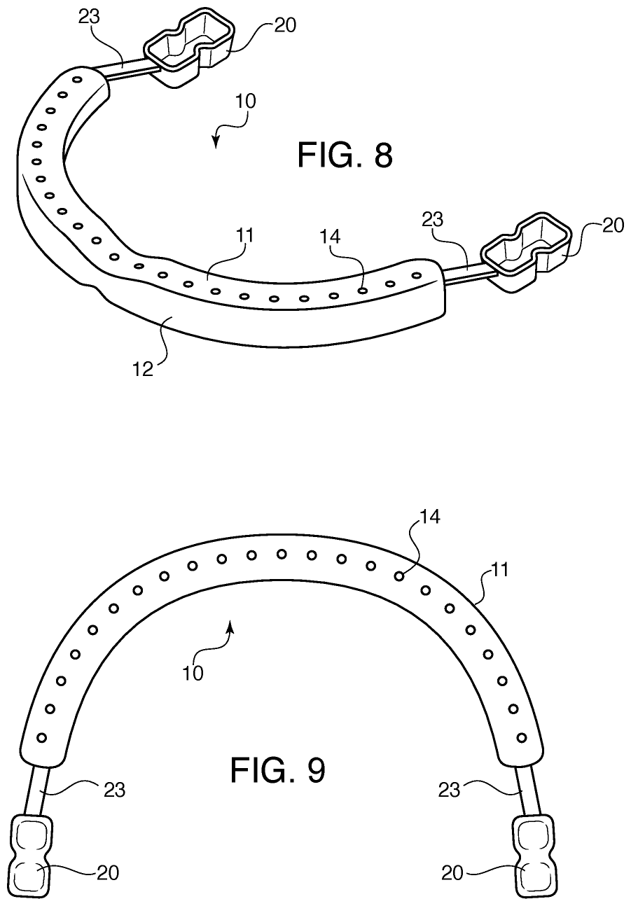

In the custom device shown in FIGS. 8 and 9, additional anchors 20 are molded with the device, to go around two of the wearer's molars, so as to encompass the molars and anchor the device in the wearer's mouth. Anchors 20 are connected to device 10 via connection legs 23, which slant downward from the muccobucal area to the molars. The device 10 can be molded precisely to the wearer's dentition to create a comfortable secure device that is firmly anchored in the mouth. In alternative embodiments (not shown), the anchors 20 can be formed to cover a single molar.

The present invention provides a simple, inexpensive and comfortable way to achieve lip enhancement without the use of injections, pharmaceuticals or surgery. It can be made for mass consumption at the retail level, or can be custom designed for each person.

Accordingly, while only a few embodiments of the present invention have been shown and described, it is obvious that many changes and modifications may be made thereunto without departing from the spirit and scope of the invention.

What is claimed is:

1. A device for enhancing the appearance of a person's lips, comprising a curved member configured to fit between the person's upper or lower lip and an adjacent gum at the front of the mouth, the curved member having a gingival portion configured to be placed on the person's gums, a top surface, and an extension connected to top surface and extending from a longitudinal edge of the top surface to overlap and extend beyond the gingival portion, the extension being configured to rest against and evert the person's upper or lower lip when the device is placed in one of the person's gums.

2. The device according to claim 1, wherein the extension is formed of a flexible material.

3. The device according to claim 1, wherein the extension has a height that is greater than a height of the gingival portion.

4. The device according to claim1, wherein the gingival portion has a thickness that is greater than a thickness of the extension.

5. The device according to claim 1, wherein the device has a plurality of indentations or perforations that are configured to increase suction of the device against the person's gums when the device is in use.

6. The device according to claim 1, wherein the indentations or perforations are located along a longitudinal edge of the device.

7. The device according to claim 1, further comprising dental impression material applied along the gingival portion or top surface.

8. The device according to claim 1, wherein a longitudinal center of the device has a height that is smaller than a height of the device on both sides of the longitudinal center.

9. The device according to claim 1, further comprising anchor portions located at each end of the device, the anchor portions being configured to engage molars of the person to anchor the device in the person's mouth.

10. The device according to claim 1, wherein the anchor portions are in molded to a shape of the person's molars.

11. A method for providing an enhanced appearance of a person's lips, comprising:
supplying a curved member configured to fit between the person's upper or lower lip and an adjacent gum at the front of the mouth, the curved member having a gingival portion configured to be placed on the person's gums, a top surface, and an extension connected to top surface and extending from a longitudinal edge of the top surface to overlap and extend beyond the gingival portion,
applying a dental impression material to the gingival portion or top surface of the curved member,
placing the curved member between the person's upper or lower lip and gum so that the dental impression material abuts the persons gum and/or muccobuccal fold, with the extension resting against and everting the person's upper or lower lip.

12. The method according to claim 1, wherein the dental impression material is polyvinyl siloxane.

13. A method for making a device for enhancing the appearance of a person's lips, comprising:
taking an impression of a person's teeth and muccobuccal region or conducting a dental scan of the person's teeth and muccobuccal region;
making a model of the person's teeth and muccobuccal region from the impression or dental scan;
adjusting the model by enlarging the muccobucal region to a desired depth;
manufacturing the device by casting a thermoformable material over the enlarged muccobucal region and trimming the thermoformable material to a desired edge size.

14. The method according to claim 13, wherein the step of casting includes casting the thermoformable material over at least on molar on each side of the mouth to create anchor points for the appliance.
